(19) United States
(12) Patent Application Publication
(10) Pub. No.: US 2003/0083748 A1
(43) Pub. Date: May 1, 2003

(54) Intervertebral fusion cage

(76) Inventors: Chong Suh Lee, Sungnam-shi (KR); Sang Soo Park, Seoul (KR); Sang Il Jung, Pyeongtaek-shi (KR); Jin Yong Lim, Seoul (KR); Sae Young Ahn, Bethesda, MD (US)

Correspondence Address: BIRCH STEWART KOLASCH & BIRCH, PO BOX 747, FALLS CHURCH, VA, 22040-0747, US

(21) Appl. No.: 09/993,618

(22) Filed: Nov. 27, 2001

(30) Foreign Application Priority Data
Oct. 30, 2001 (JP) 2001-67094

(51) Int. Cl. A61F 2/44

(52) U.S. Cl. 623/17.16

(57) ABSTRACT

A fusion cage adapted for promoting fusion of bone grafts or bone substitutes packed in the fusion cage with the adjoining vertebrae, wherein the fusion cage has a shape of cylindrical closed U of which upper and lower ends are opened such that an internal cavity is defined by a plane rear wall, both sidewalls, and a rounded front wall. An upper and lower ends of each sidewall are roundly swollen so as to secure enough contact with an upper and lower surfaces of the adjoined vertebrae.

### BACKGROUND OF THE INVENTION

(a) Field of the Invention

The present invention relates to the fusing of bone structures and, in particular, to a fusion cage using for fusing adjacent vertebral bodies or bone structures.

(b) Description of the Related Art

The vertebral column extends from the skull to the pelvis and is made up of 32˜35 individual bones (vertebrae) and intervertebral disks between adjacent vertebrae. A vertebra above and below with an intervening disk is called a motion segment. The vertebrae are divided four groups, that are cervical of 7 vertebrae, thoracic of 12 vertebrae, lumbar of 5 vertebrae, sacral of 5 vertebrae, and coccygeal of 3˜5 vertebrae, from the top of vertebral column. The sacral vertebrae are fused so as to form a sacrum as one grows up, and the coccygeal vertebrae forms one sacrum.

Many people suffer from severe back pain and the back pain has many possible etiologies including muscular, degenerative, arthritic and neurogenic causes. The treatment of this problem involves a comprehensive evaluation and an extensive trial of conservative management, including non-steroidal anti-inflammatory medications, physical therapy, manipulation, muscle relaxants and exercise programs. When the etiology of back pain has been localized to degenerative disc disease or discogenic pain and conservative therapy has failed, fusion of the spine may be the treatment of choice.

The concept of spine fusion involves the production of a solid bony fusion across a motion segment either with bone graft alone or bone graft along with some type of instrumentation. The benefit of the addition of instrumentation is to increase the fusion rate significantly.

Recently, a new and novel approach to spine fusion has been introduced. This approach centers around the use of an intervertebral cage to augment the bone graft fusion. The intervertebral cage is implanted between the vertebrae together with bone graft usually taken from the back of the pelvis or bone substitutes after removing the disc.

There are two kinds of intervertebral cages, a rectangular and horizontal cylinder types.

The rectangular type cage has broad contact surfaces relative to other types of cages, however, the disc must be completely removed for helping fusion such that it takes long time to perform cage implantation. Also, since contact portions of the cage is designed without consideration of anatomical geometries of the vertebrae, it is not expected to provide optimal conditions for cage fusion.

The horizontal cylinder type cage has some drawbacks in that the disc is partially removed for implanting the cages such that it is difficult to secure space for fusing the vertebrae. Furthermore, the threads of the cage occupy much space such that the amount of grafts to be packed therein and contact surfaces of the grafts to the vertebrae reduces, resulting in unstable fusion.

Also, since the upper and lower surfaces of the vertebrae are even but concaved and skew according to the curvature of spinal column, it is not expected that the rectangular and horizontal cylinder type cages have anatomical stable contact with the vertebrae when implanted.

### SUMMARY OF THE INVENTION

The present invention has been made in an effort to solve the above problems.

It is an object of the present invention to provide an improved intervertebral fusion cage capable of obtains stable structural support.

It is another object of the present invention to provide an intervertebral fusion cage preventing from sinking-in phenomenon, in which the cage sinks into the vertebrae, by maintaining disc heights with broad contact.

It is another object of the present invention to provide an intervertebral fusion cage capable of being implanted between the vertebrae in anatomically optimal state.

It is still another object of the present invention to provide an intervertebral fusion cage having improved biocompatibility.

To achieve the above objects, the intervertebral fusion cage of the present invention is characterized in that the cage has a shape of cylindrical closed U of which upper and lower ends are opened such that an internal cavity is defined by a plane rear wall, both sidewalls, and a rounded front wall. Upper and lower ends of each sidewall are roundly swollen so as to secure enough contact with upper and lower surfaces of the adjoined vertebrae.

### BRIEF DESCRIPTION OF THE DRAWINGS

The above and other objects and features of the instant invention will become apparent from the following description of preferred embodiments taken in conjunction with the accompanying drawings, in which:

FIG. 1 is a perspective view illustrating a fusion cage according to a the preferred embodiment of the present invention;

FIG. 2 is a front view of the fusion cage of FIG. 1;

FIG. 3a is a drawing for illustrating an angle of a tangential line of a curvature of the fusion cage with the horizontal line at the left top end of the fusion cage;

FIG. 3b is a drawing for illustrating an angle of a tangential line of the top end curvature of the fusion cage with the horizontal line at the right top end of the fusion cage;

FIG. 4a is a drawing for illustrating an angle of a tangential line of a bottom end curvature of the fusion cage with the horizontal line at the left bottom end of the fusion cage;

FIG. 4b is a drawing for illustrating an angle of tangential line of a bottom end curvature of the fusion cage with the horizontal line at the right bottom end of the fusion cage;

FIG. 5 is a top plane view illustrating the fusion cage of FIG. 1;

FIG. 6 is a side view of the fusion cage of FIG. 1 implanted between adjacent vertebrae; and

FIG. 7 is a top plane view of fusion cages of FIG. 1 as implanted.

### DETAILED DESCRIPTION OF THE PREFERRED EMBODIMENTS

A preferred embodiment of the present invention will be described hereinafter with reference to the accompanying drawings.

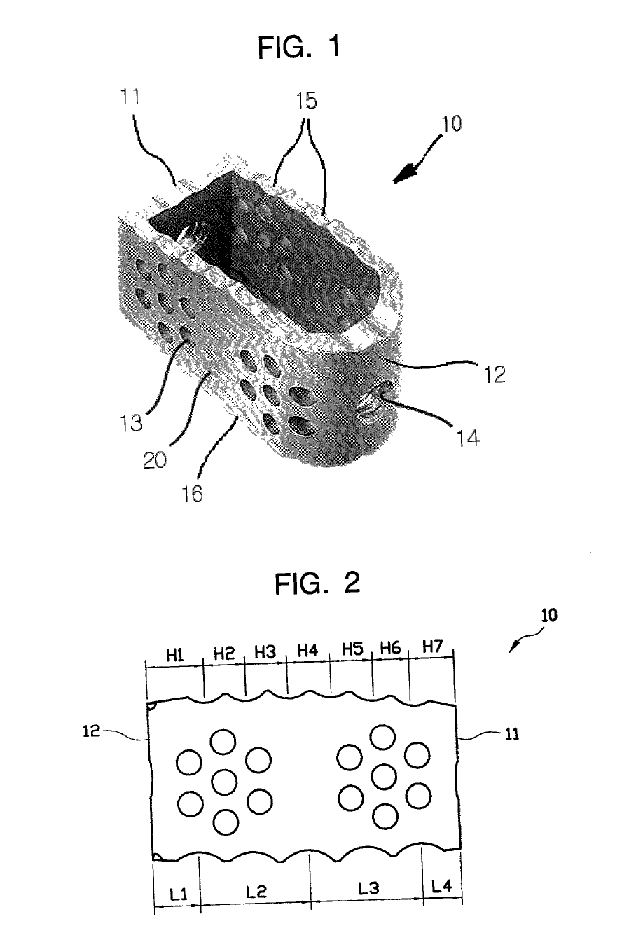

As shown in FIG. 1, the fusion cage 10 of the present invention has a shape of a cylindrical closed U of which both ends are opened such that an internal cavity is defined by a plane rear wall and both sidewalls 11 and 17 and a rounded front wall 12.

Each sidewall 20 of the cage 10 is formed such that its upper and lower ends are roundly swollen so as to secure enough contact with the upper and lower surfaces of the vertebrae and have a plurality of indents along each rounded end line.

Accordingly, a plurality of teeth having contact surfaces formed at different angles relative to an imaginary horizontal line that is perpendicular to surfaces of the walls 11, 12 and 17 of the cage.

The indents can be formed in various designs as well as in wave pattern according to the preferred embodiment of the present invention if they can prevent the cage from being slipped by twisting and bending motions.

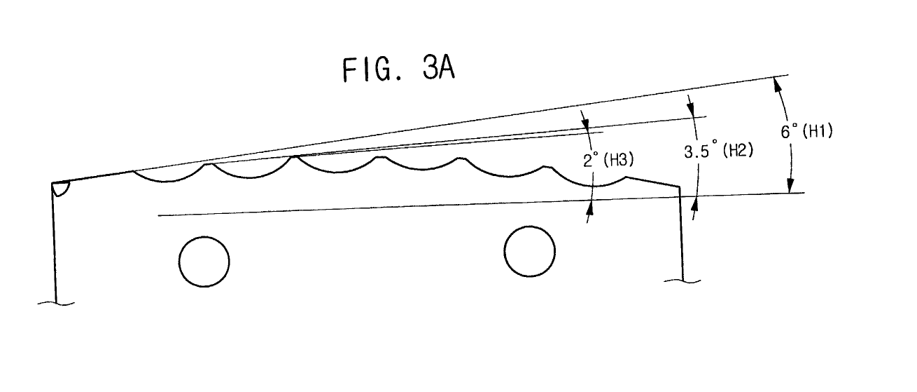

As shown in FIG. 2, the rounded upper end of the sidewall 20 is divided into a first, second, third, fourth, fifth, sixth, and seventh upper contact sections H1, H2, H3, H4, H5, H6, and H7 from the front wall 12. Among the 7 upper contact sections, the first, second, third upper contact sections H1, H2, and H3 are increasingly slanted and the fourth, fifth, sixth, and seventh upper contact sections H4, H5, H6, and H7 are decreasingly slanted from the front wall 12 to the rear wall 11.

Also, the rounded lower end of the sidewall 20 is divided 4 sections of first, second, third, and fourth lower contact sections L1, L2, L3, and L4 from the front wall 12. Among the 4 lower contact sections, the first and second lower contact sections L1 and L2 are decreasingly slanted and the third and the fourth lower contact sections L3 and L4 are increasingly slanted from the front wall 12 to the rear wall 11.

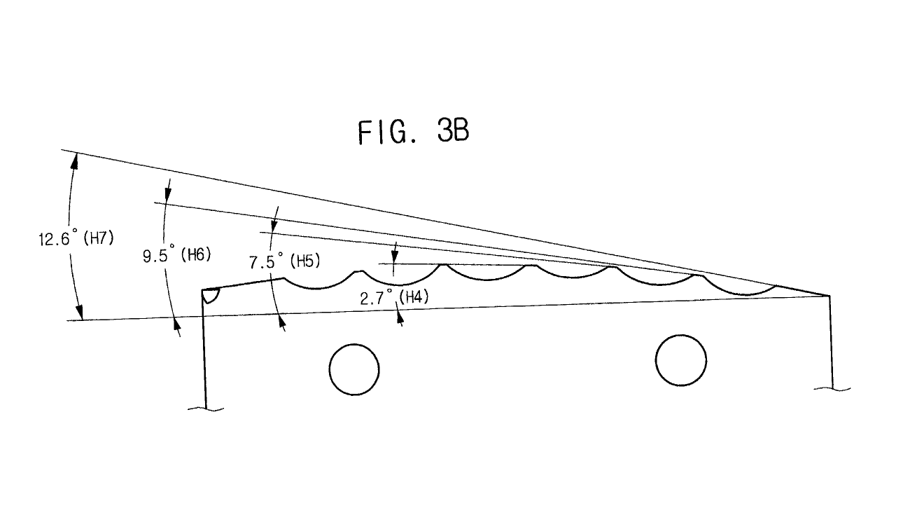

As shown in FIG. 3a, the first upper contact section H1 is formed at an angle of 5.5˜6.5°, much preferably 6°, with the horizontal line. The second upper contact section H2 is formed at an angle of 3.0˜4.0°, much preferably 3.5°, with the horizontal line. The third upper contact section H3 is formed at an angle of 2.0˜2.5°, much preferably 2°, with the horizontal line.

As shown in FIG. 3b, the fourth upper contact section H4 is formed at an angle of 2.5˜3.0°, much preferably 2.7°, with the horizontal line. The fifth upper contact section H5 is formed at an angle of 7˜8°, much preferably 7.5°, with the horizontal line. The sixth upper contact section H6 is formed at an angle of 9˜10°, much preferably 9.5°, with the horizontal line. Finally, the seventh upper contact section H7 is formed at an angle of 12˜13°, much preferably 12.6°, with the horizontal line.

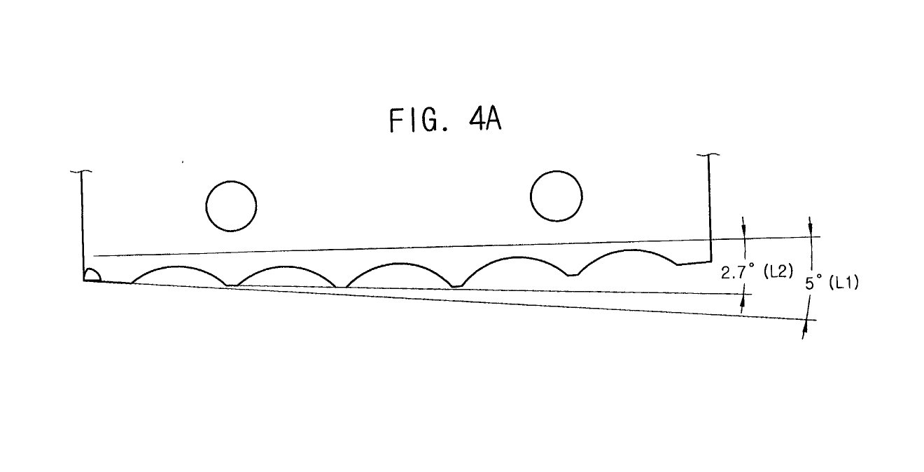

As shown in FIG. 4a, the first lower contact section L1 is formed at an angle of 4.5˜5.5°, much preferably 5°, with the horizontal line. The second lower contact section L2 is formed at an angle of 2.5˜3.0°, much preferably 2.7°, with the horizontal line.

As shown in FIG. 4b, the third lower contact section L3 is formed at an angle of 1.5˜2.5°, much preferably 2°, with the horizontal line. The four lower contact section L4 is formed at an angle of 4˜5°, much preferably 4.2°, with the horizontal line.

The fusion cage 10 is provided with a plurality of openings 13 on both the sidewalls 20 so as to prevent the grafts from being separated out of the cage 10 and facilitate the fusion of the bone grafts.

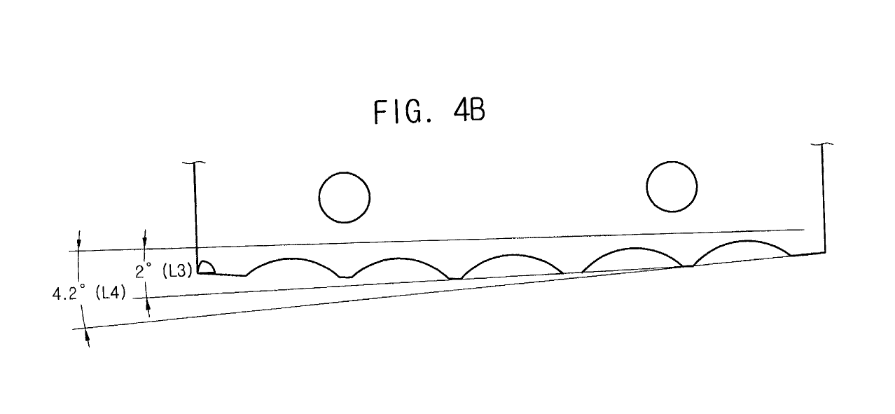

Also, the fusion cage 10 is provided with a threaded hole 14 on each of the rear and front walls 11 and 12 that are coaxially formed and a pair of vertical grooves 17 (see FIG. 5) such that the cage 10 is connected with an implant assistant device (not shown) for assisting the implantation of the cage 10.

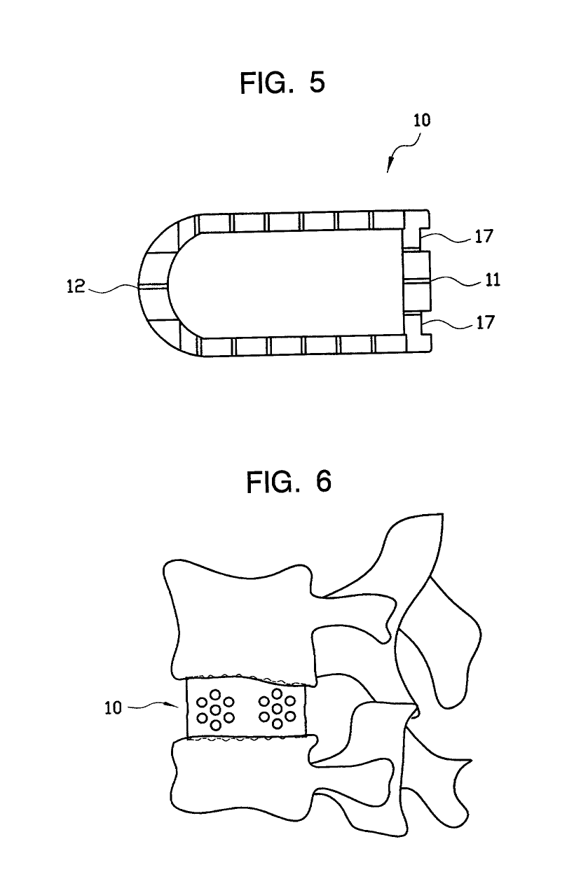

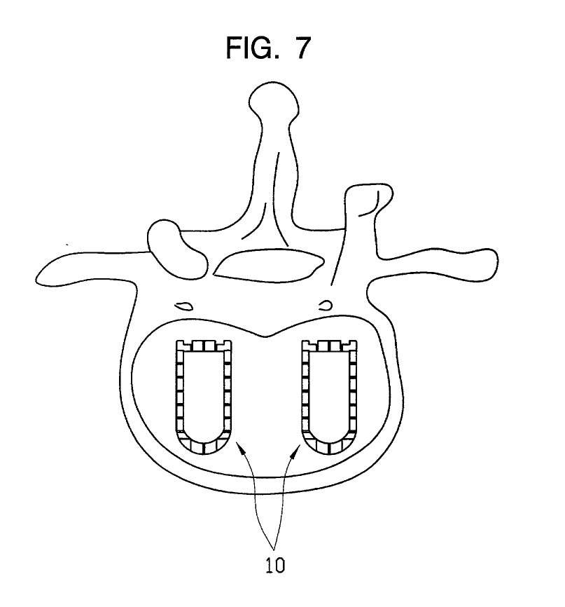

As shown in FIG. 6 and FIG. 7, the fusion cage 10 is inserted and appropriately placed between two vertebrae such that the upper and lower ends of the cage 10 are contacted with the upper and lower surfaces of the vertebrae in anatomically optimal osculating curves.

The fusion cage 10 of the present invention is made out of pure titanium or titanium alloy such as Ti-6Al-4V-ELI.

Also, the fusion cage 10 can be manufactured with materials that can be harmlessly absorbed and autolyzed in the human body such as Poly L-Lactic Acid (PLLA), Poly Lactic-co-glycolic Acid (PLGA), Poly Glycolic Acid (PGA).

As described above, the intervertebral fusion cage of the present invention is designed so as to have anatomically optimal osculating curves at its contact portions such that the fusion cage can be stably and optimally implanted between the vertebrae according to the spine curvature.

The fusion cage of the present invention is provided with the plurality of openings on its side walls, which facilitate the fusion of the bone grafts packed in the cage.

Also, since the fusion cage of the present invention is made out of pure titanium, titanium alloy or materials that can be harmlessly absorbed and autolyzed in the human body, it offers an excellent fusion rate with potentially less postoperative pain.

Furthermore, the fusion cage maintains disc height with broad contact so as to prevent from the sinking-in phenomenon in which the cage sinks into the vertebrae.

While this invention has been described in connection with what is presently considered to be the most practical and preferred embodiment, it is to be understood that the invention is not limited to the disclosed embodiments, but, on the contrary, is intended to cover various modifications and equivalent arrangements included within the spirit and scope of the appended claims.

What is claimed is:

1. A fusion cage adapted for promoting fusion of bone grafts or bone substitutes packed in the fusion cage with the adjoining vertebrae has a shape of cylindrical closed U of which upper and lower ends are opened such that an internal cavity is defined by a plane rear wall, both sidewalls, and a rounded front wall.

2. A fusion cage of claim 1 wherein an upper and lower ends of each sidewall are roundly swollen so as to secure enough contact with an upper and lower surfaces of the adjoined vertebrae.

3. A fusion cage of claim 2 wherein the upper and lower ends of the sidewall are provided with a plurality of indents along each rounded end line such that a plurality of teeth having contact surfaces formed at different angles relative to an imaginary horizontal line that is perpendicular to surfaces of the walls of the cage.

4. A fusion cage of claim 3 wherein the rounded upper end of the sidewall is divided into first, second, third, fourth, fifth, sixth, and seventh upper contact sections from the front wall.

5. A fusion cage of claim 4 wherein the first, second, and third upper contact sections are increasingly slanted from the front wall.

6. A fusion cage of claim 5 wherein the fourth, fifth, sixth, and seventh upper contact sections are decreasingly slanted to the rear wall.

7. A fusion cage of claim 6 wherein the first, second, third, fourth, fifth, sixth, and seventh upper contact sections are formed at respective ranges of 5.5˜6.5°, 3.0˜4.0°, 2.0˜2.5°, 2.5˜3.0°, 7˜8°, 9˜10° 9.5°, and 12˜13° with the horizontal line.

8. A fusion cage of claim 6 wherein the first, second, third, fourth, fifth, sixth, and seventh upper contact sections are formed at respective angles of 6°, 3.5°, 2°, 2.7°, 7.5°, 9.5°, and 12.6°.

9. A fusion cage of claim 3 wherein the rounded lower end of the side wall is divided into a first, second, third, and fourth lower contact sections from the front wall.

10. A fusion cage of claim 9 wherein the first and second lower contact sections are decreasingly slanted from the front wall.

11. A fusion cage of claim 10 wherein the third and fourth lower contact sections are increasingly slanted to the rear wall.

12. A fusion cage of claim 11 wherein the first, second, third, and fourth lower contact sections are formed at respective ranges of 4.5˜5.5°, 2.5˜3.0°, 1.5˜2.5°, and 4.0˜5.0° with the horizontal line.

13. A fusion cage of claim 11 wherein the first, second, third, and fourth lower contact sections are formed at respective angles of 5.0, 2.7, 2.0, and 4.2 with the horizontal line.

14. A fusion cage of claim 1 wherein the sidewall is provided with a plurality of openings so as to prevent the bone grafts from being separated out of the cage and facilitate the fusion of the fusion.

15. A fusion cage of claim 14 wherein the openings are arranged in a radial shape.

16. A fusion cage of claim 1 wherein the fusion cage is provided with a threaded hole on each of the rear and front walls that are coaxially formed such that the fusion cage is connected with an implant assistant device for assisting implantation of the fusion cage.

17. A fusion cage of claim 16 wherein the fusion cage is provided with a pair of vertical grooves for helping connection of the implant assistant device with the fusion cage.
